(19) United States
(12) Patent Application Publication
(10) Pub. No.: US 2021/0400871 A1
(43) Pub. Date: Dec. 30, 2021

(54) AGRICULTURAL HARVESTING MACHINE

(71) Applicant: CLAAS Selbstfahrende Erntemaschinen GmbH, Harsewinkel (DE)

(72) Inventors: Frédéric Fischer, Arnsberg (DE); Marvin Bather, Steinhagen (DE); Maik Heufekes, Munster (DE); Dennis Neitemeier, Lippetal (DE); Johann Witte, Frondenberg (DE)

(73) Assignee: CLAAS Selbstfahrende Erntemaschinen GmbH, Harsewinkel (DE)

(21) Appl. No.: 17/355,922

(22) Filed: Jun. 23, 2021

(30) Foreign Application Priority Data
Jun. 29, 2020 (DE) 102020117069.6

(51) Int. Cl. A01D 41/127; A01D 61/02; G01N 33/02; G01N 21/359; G06T 7/00; H04N 9/04

(52) U.S. Cl. CPC A01D 41/1277; A01D 61/02; G01N 33/025; G01N 21/359; G06T 2207/10048; H04N 9/04; G06T 2207/30188; G06T 2207/10024; G06T 7/0002

(57) ABSTRACT

An agricultural harvesting machine, with at least one work assembly and a monitoring assembly, is disclosed. The agricultural harvesting machine transports harvested material in a harvested material flow along a harvested material transport path. The monitoring assembly includes an optical measuring system positioned on the harvested material transport path and an evaluation device configured to determine at least one harvested material parameter. The optical measuring system includes a first optical sensor that senses spatially-resolved image data indicative of visible light in a first section and second optical sensor that senses image data indicative of invisible light in a second section that at least partly overlaps the first section. The evaluation device correlates the image data for the overlapping section from the first optical sensor and from the second optical sensor and determines, based on the correlation, at least one harvested material parameter.

### CROSS REFERENCE TO RELATED APPLICATION

This application claims priority under 35 U.S.C. § 119 to German Patent Application No. DE 102020117069.6 filed Jun. 29, 2020, the entire disclosure of which is hereby incorporated by reference herein.

### TECHNICAL FIELD

The invention relates to an agricultural harvesting machine that includes an optical measuring system to generate optical data and an evaluation device to determine, based on the optical data, at least one harvested material parameter.

### BACKGROUND

This section is intended to introduce various aspects of the art, which may be associated with exemplary embodiments of the present disclosure. This discussion is believed to assist in providing a framework to facilitate a better understanding of particular aspects of the present disclosure. Accordingly, it should be understood that this section should be read in this light, and not necessarily as admissions of prior art.

Agricultural harvesting machines, such as, for example, combines and forage harvesters, are configured to harvest a crop from a field and to process the harvested material thus obtained by using a series of work assemblies. In principle, the crop may already have different qualities. To an extent, however, the quality of the crop may still be influenced by the harvesting process. In particular, a great deal of importance may be ascribed to the separation of grain components and non-grain components. In this regard, it may be important to determine the quality, or some other type of harvesting parameter, of the harvested material in order to directly correct the harvesting process and/or to record as information and for documentation.

U.S. Pat. No. 9,648,807 B2 (incorporated by reference herein in its entirety) discloses an agricultural harvesting machine that transports the harvested material in a harvested material flow along a harvested material transport path through the harvesting machine. The agricultural harvesting machine includes a control assembly that has an optical measuring system arranged or positioned on the harvested material transport path to analyze the composition and/or contents of the harvested material. The optical measuring system has an optical sensor for the spatially-resolved recording of visible light of a visible wavelength range within a field of vision. In a measuring routine, the optical measuring system records spatially-resolved image data from the optical sensor within the visible wavelength range that depict the harvested material within a section of the harvested material flow. The optical sensor in the agricultural harvesting machine may comprise a commercially-available camera. Moreover, the harvesting machine has an evaluation device for determining a harvested material parameter by using image data from the optical sensor.

Optical sensors or other sensors may be used in order to determine harvested material parameters that cannot be optically determined within the visible range.

### DESCRIPTION OF THE FIGURES

The present application is further described in the detailed description which follows, in reference to the noted drawings by way of non-limiting examples of exemplary implementation, in which like reference numerals represent similar parts throughout the several views of the drawings, and wherein:

FIG. 1 shows a schematic side view of a combine as a proposed agricultural harvesting machine; and

FIG. 2 illustrates a schematic side view of a grain elevator of the combine from FIG. 1 with a proposed optical measuring system.

### DETAILED DESCRIPTION

As discussed in the background, agricultural harvesting machines may analyze the harvested material flow. However, that the harvested material in the harvested material flow may not be homogeneous in terms of location or time. In this regard, there may be a need to optimize the analytical systems with respect to their spatial resolution and/or their time resolution.

Thus, in one or some embodiments, an agricultural harvesting machine is disclosed that may precisely determine one or more harvested material parameters, such as in an optimal manner

In one or some embodiments, the system uses a combination (such as a combination resident on the agricultural harvesting machine itself) a first optical sensor with a second optical sensor, wherein the first optical sensor is different in one or more aspects from the second optical sensor (e.g., a camera generating a camera-based measurement within the visible wavelength range with an additional optical measurement performed by a non-camera-based device). This additional optical measurement may occur within a different wavelength range from the camera, such as within the invisible wavelength range. The system may then analyze a section, such as an overlapping section (e.g., a matching overlapping section for both the first optical sensor and the second optical sensor) of data generated by both optical sensors indicative of the harvested material in the harvested material flow. In this way, the data from the two optical sensors are not used for separate determinations (e.g., determining separate harvested material parameters); rather, the data from the two optical sensors are analyzed in combination in order to jointly determine at least one harvested material parameter.

In particular, in one or some embodiments, the optical measuring system includes a second optical sensor for recording invisible light from an invisible wavelength range in a second field of vision; within the measuring routine, the optical measuring system records image data from the second optical sensor within the invisible wavelength range that depict the harvested material within a second section of the harvested material flow; the first section and the second section at least partially overlap in an overlapping section; and in an analysis routine, the evaluation device analyzes the data generated by the first optical sensor and the second optical sensor in combination (e.g., correlates the image data from the first optical sensor on the overlapping section and from the second optical sensor on the overlapping section with each other) and thereby determines the harvested material parameter based on the analysis (e.g., based on the correlation).

In one or some embodiments, the first optical sensor may be designed as a camera, such as an RGB camera, and/or the second optical sensor may be designed as, for example, a near-infrared spectrometer. Near-infrared data may be prone to fluctuation. These fluctuations frequently are not based on changes in the harvested material parameter, but rather on changes in the measuring conditions, other harvested material parameters, etc. Many of these fluctuations are, in turn, detectable within the visible wavelength range. Frequently, these fluctuations are also locally very limited. Through the disclosed overlapping section, the determination of one or more harvested material parameters may become significantly more precise or, in some cases, made possible in the first place based on the analysis of the overlapping section, thereby reducing the effect of these fluctuations.

In one or some embodiments, the optical measuring system may include a housing and a transparent window. The first optical sensor and second optical sensor may be arranged or positioned at least partly within the housing (such as one or both entirely within the housing). Using the common housing and another beam guidance system in the housing of the light to measured, the overlapping of the first section and the second section may be ensured or configured in a robust and precise manner.

The harvested material parameter may be determined by the evaluation device determining the harvested material parameter from the image data of the second sensor and correcting it with the image data from the first optical sensor. For example, the evaluation device may be configured to determine the harvested material parameter relating to at least a part of the overlapping section from the image data of the second optical sensor such that the evaluation device determines a correction value (e.g., a correction factor) from the image data of the first optical sensor and corrects the harvested material parameter using the correction factor.

Various harvested material parameters are contemplated. In particular, the harvested material parameter and/or the correction value may relate to any one, any combination or all of: a grain portion; a broken grain portion; a non-grain portion (e.g., any one, any combination, or all of: a portion of awns; unthreshed components; straw; stem parts; or silage); or a content of the harvested material (e.g., any one, any combination, or all of: a protein content; starch content; a grain moisture). Alternatively, or in addition, the correction value may depict a measurability of the first section and/or second section by the second optical sensor.

In one or some embodiments, the first optical sensor may distinguish between at least two visible wavelength ranges with spatial resolution. This may be performed by the RGB camera. It is also contemplated that other types of sensors may perform this function. In particular, the first optical sensor may be configured for the spatially-resolved recording of visible light from at least two, such as at least three, different visible wavelength ranges within the first field of vision, and/or the first optical sensor is configured for the spatially resolved recording of visible light for the entire visible wavelength range within the first field of vision. Accordingly, one or more pieces of information on the harvested material may be detected within different ranges (such as different ranges that at least partly overlap or different ranges that are mutually exclusive) of color.

In one or some embodiments, the first optical sensor may comprise a line scan camera or area scan camera with sensor elements, and the sensor elements may each record locally different pixels of the first field of vision that are at a distance from each other.

In one or some embodiments, the second optical sensor may have at most 1000 (e.g., at most 500; at most 100; at most 10; at most 0) sensor elements that record locally different pixels, and the second optical sensor comprises a non-spatially resolved spectrometer. Alternatively, or in addition, at least one sensor element of the second optical sensor may be designed to record or sense invisible light from at least one wavelength range from any one, any combination, or all of the following ranges: 1000 nm; 1200 nm to 1800 nm; 2000 nm; 2100 nm; 2200 nm; 2300 nm; 2400 nm; 2500 nm; 2200 to 2400 nm; 2100 nm to 2500 nm; a portion of the near-infrared spectrum (e.g., wavelength range where wavelengths are greater than 700 nanometers); a portion of the near ultraviolet spectrum or an ultraviolet range ((e.g., wavelength range where wavelengths are less than 380 nanometers); or invisible light from at least one wavelength range or at least one wavelength comprising any one, any combination, or all of: 300 nm; 330 nm; 350 nm; or 380 nm.

In one or some embodiments, the first section and the second section at least partially overlap each other. In a particular embodiment, there is no time component in this overlap; rather, the overlap relates to one and the same location. Accordingly, influences from dynamic changes in the harvested material or harvested material flow can be reduced or minimized. Further, in one or some embodiments, the first section covers at least 50%, at least 90%, at least 95%, at least 99%, or at 100% of the second section (e.g., so the first section and the second section are coextensive).

In one or some embodiments, the agricultural harvesting machine may include a transparent window through which light may be transmitted to one or both of the first optical sensor or the second optical sensor. Thus, the transparent window may be arranged relative to the agricultural harvesting machine, and any other preferred arrangement of the optical measuring system and/or its parts relative to the agricultural harvesting machine. In a particular embodiment, the transparent window is adjacent to and/or borders the harvested material transport path. The agricultural harvesting machine may include a grain elevator, with any one, any combination, or all of the first optical sensor; the second optical sensor; the transparent window; the light source; or the housing being arranged on (e.g., arranged under or over) the grain elevator.

In one or some embodiments, a method includes operating an agricultural harvesting machine so that a crop is harvested and harvested material is processed, wherein the measuring routine is performed using the optical measuring system, and wherein the analysis routine is performed using the evaluation device. Reference is made to all statements regarding functionality of the disclosed agricultural harvesting machine.

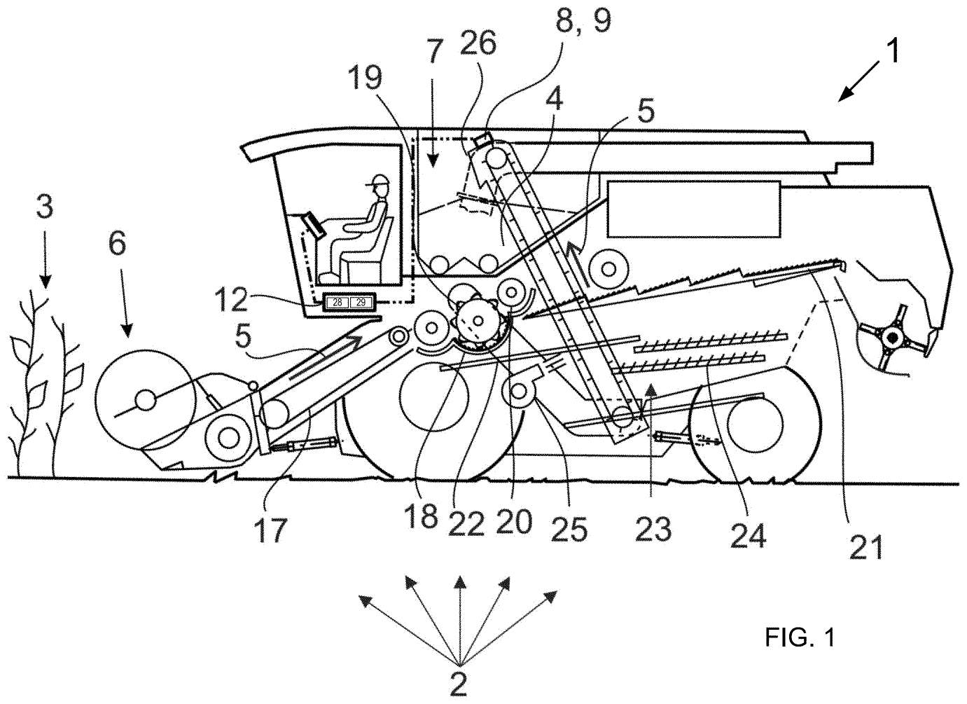

Referring to the figures, the agricultural harvesting machine shown in FIG. 1, which may comprise a combine 1, has at least one work assembly 2 for harvesting a crop 3 and for processing harvested material 4 of the crop 3. Alternatively, the agricultural harvesting machine may comprise a forage harvester. While the harvesting machine is operating, the harvested material 4 is transported in a harvested material flow along a harvested material transport path 5 through the harvesting machine.

While being transported through the harvesting machine, the harvested material 4 forms a harvested material flow. In one or some embodiments, the term “harvested material flow” comprises the flow of the plant parts of the crop 3 to be processed on the harvested material transport path 5. In this case, this harvested material transport path 5 begins, especially in a combine 1, with a cutting unit 6 and proceeds to the grain tank 7. In one or some embodiments, the harvested material flow may be divided into a main harvested material flow and smaller partial harvested material flows. The term “main harvested material flow” then identifies the part of the harvested material flow that contains the majority of the harvested material 4 relative to the overall harvested material transport path 5. Partial harvested material flows on a smaller scale that branch off, such as for analytical purposes, are not included.

The agricultural harvesting machine has a monitoring assembly 8 that has an optical measuring system 9 arranged or positioned on the harvested material transport path 5 for analyzing the harvested material 4. The analysis of the harvested material may include an analysis of the composition (e.g., any one, any combination, or all of share of undamaged grain, share of broken grain, or share of non-grain, etc.) of the harvested material in the harvested material flow, and/or an analysis of the contents (e.g., any one, any combination, or all of moisture content, protein content, starch content, sugar content, or fat content, etc.) of certain plant components in the harvested material flow, such as the grain.

The optical measuring system 9 has a first optical sensor 10 for the spatially-resolved recording of visible light from a visible wavelength range within a first field of vision 11. Given the definitions of the visible wavelength range that sometimes differ slightly from each other, in one or some embodiments, visible light may comprise a wavelength range at least partly between 380 nm and 780 nm (e.g., 380 nm to 780 nm).

In one or some embodiments, the term “field of vision” relates to the three-dimensional space from which light may contact the particular optical sensor through the corresponding optical system. The field of vision in general parlance may also be frequently expressed as the English term “field-of-view”. The term “spatially resolved” may mean that the field of vision of the particular optical sensor is divided into several partial fields of vision that can be distinguished from each other by measuring. In one or some embodiments, the particular optical sensor used therefore has at least two pixels which at least partially have a different partial field of vision. A pixel is a two-dimensional depiction of a partial field of vision. In general parlance, picture elements are frequently also identified by the English term pixel. As can be seen, a broad definition of an optical system may be used that is directed to more than just the visible wavelength range.

In a measuring routine, the optical measuring system 9 may record spatially-resolved image data from the optical sensor 10 within the visible wavelength range that depict the harvested material 4 within a first section A₁ of the harvested material flow. The term “image data” in this case may generally relate to sensor data generated by an optical sensor. The section of the harvested material 4 is the part of the harvested material 4 visible to the optical sensor at the time of recording or sensing. In particular, the section of the harvested material 4 relates to the uncovered part of the harvested material 4 located within the field of vision.

The harvesting machine moreover includes an evaluation device 12 for determining one or more harvested material parameters, such as relating to the composition and/or contents of the harvested material 4. The evaluation device 12 may comprise any type of computing functionality, such as at least one processor 28 (which may comprise a microprocessor, controller, PLA, or the like) and at least one memory 29 in order to perform the functions described herein, such as, for example, analyzing sensor data and/or determining one or more harvested material parameters. The memory may comprise any type of storage device (e.g., any type of memory). Though the processor 28 and memory 29 are depicted as separate elements, they may be part of a single machine, which includes a microprocessor (or other type of controller) and a memory.

The processor 28 and memory 29 are merely one example of a computational configuration. Other types of computational configurations are contemplated. For example, all or parts of the implementations may be circuitry that includes a type of controller, including an instruction processor, such as a Central Processing Unit (CPU), microcontroller, or a microprocessor; or as an Application Specific Integrated Circuit (ASIC), Programmable Logic Device (PLD), or Field Programmable Gate Array (FPGA); or as circuitry that includes discrete logic or other circuit components, including analog circuit components, digital circuit components or both; or any combination thereof. The circuitry may include discrete interconnected hardware components or may be combined on a single integrated circuit die, distributed among multiple integrated circuit dies, or implemented in a Multiple Chip Module (MCM) of multiple integrated circuit dies in a common package, as examples.

In one or some embodiments, the optical measuring system 9 includes a second optical sensor 13 configured to record or sense invisible light at least partly from an invisible wavelength range in a second field of vision 14. In one or some embodiments, the particular optical sensor 10, 13 does not exclusively have to record or sense visible or invisible light. Certain overlaps are contemplated. However, in one or some embodiments, more than 50% of the recorded light of the wavelength range is to be recorded or sensed of the particular optical sensor 10, 13 originates from the visible or invisible range. In one or some embodiments, even at least 90%, or at least 99%, or 100%, originates from the particular wavelength range.

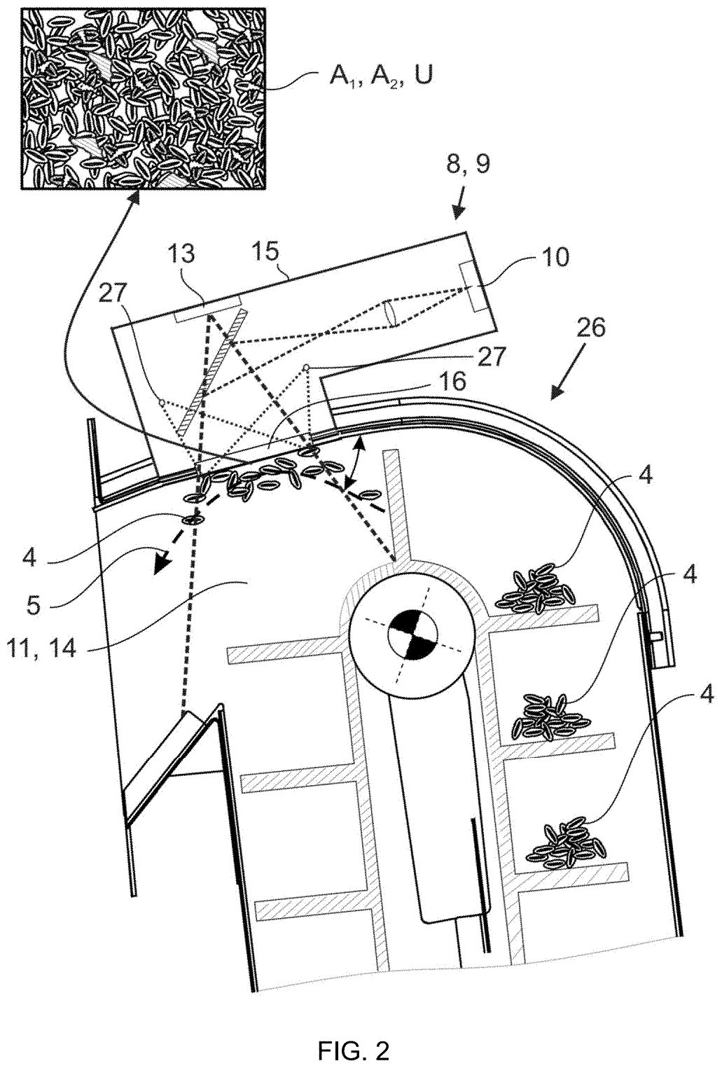

FIG. 2 illustrates the first optical sensor 10 and the second optical sensor 13 separated by a semitransparent minor. This configuration is disclosed merely as an example.

In the measuring routine, the optical measuring system 9 records image data from the second optical sensor 13 within the invisible wavelength range that depict the harvested material 4 within a second section A₂ of the harvested material flow.

In one or some embodiments, the first and second section A₁, A₂ at least partially overlap in an overlapping section U, and in an analysis routine (which may comprise software executed by a processor, discussed below), the evaluation device 12 is configured to correlate the image data from the first optical sensor 10 on the overlapping section and from the second optical sensor 13 on the overlapping section U with each other and thereby (based on the correlation) determines the one or more harvested material parameters.

The overlapping section U may be resolved or formed temporally and/or spatially. For example, the first optical sensor 10 and the second optical sensor 13 may be arranged or positioned spatially offset from each other. Since in this case, the evaluation device 12 is aware of a speed of the harvested material flow (e.g., the evaluation device may access a pre-determined speed of the harvested material flow or may receive as input via another sensor (not shown) the speed of the harvested material flow), the overlapping section U may therefore be calculated by a delay in the recording or the sensing by the respective sensors. Alternatively or in addition, the overlapping section U may be formed spatially (e.g., by an overlap of the first field of vision 11 and the second field of vision 14).

In one or some embodiments, the harvested material transport path 5 may always run at least partially (or only partially) through the first and through the second field of vision 11, 14. In this case, the overlapping section U relates to the harvested material flow and in particular the main harvested material flow. A static measurement is, however, also contemplated.

Various correlations performed by the evaluation device are contemplated. In this regard, the term “correlate” is to be understood broadly in this context. In one or some embodiments, the image data from the first optical sensor 10 and the second optical sensor 13 may be used together or analyzed in combination (e.g., any one, any combination, or all of an additive sense, a subtractive sense, a difference sense, or a sameness sense) to determine the harvested material parameter and, in so doing, are processed together to determine the harvested material parameter. To accomplish this, experimental dependencies may, for example, be determined between the image data.

In one or some embodiments, the first optical sensor 10 comprises a camera, such as an RGB camera. An RGB camera comprises a color camera that has a least one red, at least one green, at least one blue color channel. The RGB camera may therefore record three distinguishable wavelength ranges. It is normally the case that the sensor elements of the camera are distributed to the wavelength ranges using a color filter, such as in the manner of a Bayer pattern.

In one or some embodiments, the second optical sensor 13 comprises a spectrometer, such as a near-infrared spectrometer. A near-infrared spectrometer may distinguish at least two distinguishable wavelength ranges in the near-infrared range and determine an intensity of the light in this wavelength range. In one or some embodiments, the second optical sensor 13 comprises an optical spectrometer.

In one or some embodiments, the near-infrared wavelength range in this case may be defined as running from 780 nm to 3000 nm. The near-ultraviolet wavelength range in this case may be defined as running from 315 nm to 380 nm.

In one or some embodiments, the optical measuring system 9 has a housing 15. In one or some embodiments, the first optical sensor 10 and the second optical sensor 13 are arranged or positioned at least partly in the housing 15 (such as one or both entirely in the housing 15). Thus, in one embodiment, the entire sensor 10, 13 may be within housing 15. Alternatively, only a part of the particular optical sensor 10, 13 may be arranged in the housing 15; in particular, all light-receptive sensor elements of the particular optical sensor 10, 13 may be arranged or positioned in the housing 15.

The optical measuring system 9 may have at least one transparent window, such as a transparent window 16. In one or some embodiments, the light recorded by the first and second optical sensor 10, 13 that proceeds from the harvested material 4 (e.g., from the particular sections A₁, A₂ of the harvested material 4) traverses through the transparent window 16 and from the transparent window 16 to the first and second optical sensor 10, 13 (which may be completely within the housing 15). This is illustrated in FIG. 2. In one or some embodiments, the transparent window 16 is part of the housing 15. In one or some embodiments, the transparent window 16 may come into contact with the harvested material 4.

The procedure for determining the harvested material parameter may be such that the evaluation device 12 determines a harvested material parameter relating to the overlapping section U from the image data of the second optical sensor 13 such that the evaluation device 12 determines a correction value, such as a correction factor, from the image data of the first optical sensor 10 and corrects the harvested material parameter using the correction factor. The harvested material parameter may therefore be determined in this case especially for the overlapping section U. This is particularly relevant when the harvested material parameter is inhomogeneous over the harvested material flow.

The harvested material parameter and/or the correction value may relate to any one, any combination, or all of: a grain portion; a broken grain portion; a non-grain portion (e.g., any one, any combination, or all of: a portion of awns; unthreshed components; straw; or a content of the harvested material 4 (e.g., any one, any combination, or all of: a protein content; a starch content; or a grain moisture). The correction value may depict some aspect of the first and/or second section A₁, A₂ (e.g., a measurability of the first and/or second section A₁, A₂ by the second optical sensor 13). For example, it is contemplated for a harvested material parameter to be easily determinable using the sensor data from the second optical sensor 13, but susceptible to a disruptive factor such as a non-grain share that in turn may be easily determinable using the first optical sensor 10. If the influence of the non-grain portion on the overall measuring result from the second sensor 13 is then approximately known, its measured value may be correspondingly corrected, and the measurability of the section may therefore be taken into account. The term “measurability” relates to the issue of how much a measurement of the harvested material parameter in the particular section A₁, A₂ by the second optical sensor 13 is completely or only partially reasonably possible.

In one or some embodiments, various harvested material parameters and disruptive factors in different wavelength ranges may be determined with different precision and mutual interference using the two optical sensors 10, 13, whereby the evaluation device may use an equation system (which may be manifested in one or more equations) reflective of the different factors together forming the measuring result of the particular wavelength range. Given a sufficient degree of certainty, the evaluation device may then solve this equation system. In one or some embodiments, different wavelengths may have a different penetration depth into the harvested material 4 which allows correspondingly different harvested material parameters to be measured.

In one or some embodiments, with respect to the example of the RGB camera, the first optical sensor 10 may be configured for the spatially resolved recording of visible light from at least two, such as at least three different visible wavelength ranges within the first field of vision 11. The first optical sensor 10 may, in addition or alternatively, be configured for the spatially resolved recording of visible light for the entire visible wavelength range within the first field of vision 11. The different wavelength ranges may be recorded simultaneously or sequentially. Simultaneous recording may be achieved by means of a Bayer pattern, light refraction, beam division, etc. Sequential recording may be achieved passively by changing filters in the manner of a filter wheel, or actively by sequential illumination in the different wavelength ranges.

The first optical sensor 10 may be designed as a line scan camera or area scan camera with sensor elements. The sensor elements may each record locally different pixels of the first field of vision 11 that are, for example, at a distance from each other. In one or some embodiments, the pixels do not overlap. In one or some embodiments, the first optical sensor 10 has at least 1,000 sensor elements, or at least 10,000 sensor elements that each record the same wavelength range. The first optical sensor 10 therefore has for example at least 1 million sensor elements provided with a green filter. In addition or alternatively, the sensor elements may be arranged flat, in particular on a common sensor chip. The sensor elements can be designed using known technologies, for example as CCD sensor elements, or CMOS sensor elements, or InGaAs sensor elements. Depending on the method of counting, they may form individually or in groups, such as of four, that which is normally also identified by the English term “pixel”.

In summary, the first optical sensor 10 may provide for the spatially resolved recording of at least one, such as fewer spectral ranges. In one or some embodiments, the spatial resolution may also be assigned a spectral resolution so that a matrix results from the spatial resolution and spectral resolution. Merely by way of example, this is known to be the case with a Bayer pattern.

For the second optical sensor 13, in one or some embodiments, the second optical sensor 13 is different in one or more aspects to the first optical sensor 10. In one or some embodiments, the second optical sensor 13 may have lesser spatial resolution than the first optical sensor 10. Regardless, it is contemplated that the second optical sensor 13 may have any desired spectral resolution.

The second optical sensor 13 may be configured to record invisible light comprising (or consisting of) at least two distinguishable wavelength ranges, such as at least 100 distinguishable wavelength ranges, such as at least 1,000 distinguishable wavelength ranges, such as at least 5,000 distinguishable wavelength ranges, such as at most 20,000 distinguishable wavelength ranges, or such as at most 10,000 distinguishable wavelength ranges. The second optical sensor 13 in addition or alternatively may have at most 1000 sensor elements, such as at most 500 sensor elements, such as at most 100 sensor elements, such as at most 10 sensor elements, such as no sensor elements that record locally different pixels. In one or some embodiments, the second optical sensor 13 is designed as a non-spatially resolved spectrometer. It can therefore be provided that the second optical sensor 13 only has just one local pixel per spectral pixel.

In one or some embodiments, the first optical sensor 10 has at least twice as many sensor elements, such as at least four times as many sensor elements, such as at least ten times as many sensor elements, such as at least 100 times as many sensor elements as the second optical sensor 13.

In this case, at least one sensor element of the second optical sensor 13 is designed to record invisible light from at least one wavelength range (and/or wavelength), any combination wavelength ranges (and/or wavelengths), or all wavelength ranges (and/or wavelengths) comprising: 1000 nm and/or 1200 nm to 1800 nm and/or 2000 nm, and/or comprising 2100 nm and/or 2200 nm and/or 2300 nm and/or 2400 nm and/or 2500 nm, and/or comprising 2200 to 2400 nm, and/or comprising 2100 nm to 2500 nm, and/or comprising a portion of the near-infrared spectrum, and/or comprising a portion of the near ultraviolet spectrum.

Moreover, in one or some embodiments, at least one sensor element of the second optical sensor 13 may be designed to record invisible light from at least one wavelength range comprising 300 nm, and/or 330 nm, and/or 350 nm, and/or 380 nm.

In one or some embodiments, another wavelength range of interest within the field of agriculture is 405 nm. At this wavelength, in particular the recognition of the broken grain portion is particularly recognized. In this regard, in one or some embodiments, the second optical sensor 13 is designed to record light from a wavelength range comprising 405 nm in addition to an invisible wavelength range.

In one or some embodiments, the distinguishable wavelength ranges of the second optical sensor 13 may be wider than 0.1 nm, such as wider than 1 nm, such as wider than 4 nm and/or less wide than 100 nm, such as less wide than 50 nm, such as less wide than 10 nm, such as less wide than 5 nm.

In one or some embodiments, the first section A₁ may cover at least 50%, such as at least 90%, such as at least 95%, such as at least 99%, or such as 100% of the second section A₂. The coverage in this case is meant in terms of time and/or location.

Alternatively with respect to coverage in terms of time, the first section A₁ and the second section A₂ may overlap at their edges or are at a distance from each other of at most one meter, such as at most one-half meter, or such as at most ten centimeters.

With the combine 1 illustrated, the work assembly(ies) 2 include the previously mentioned cutting unit 6 and an auger 17 connected thereto from which the harvested material flow is transferred to a threshing unit 19 surrounded by a threshing concave 18. Using a deflection drum 20, the harvested material flow enters into a separating device 21 designed in this case as a separating rotor in which freely mobile grains of the harvested material flow are deposited in a lower area. From here, the harvested material flow passes via a returns pan 22 to a cleaning device 23 that, as shown here, comprises (or consists of) several screening levels 24 and a blower 25. From here, the grain elevator 26 finally may guide the harvested material flow into the grain tank 7. In one or some embodiments, all of these work assemblies 2 may contribute to the processing of the harvested material 4.

In one or some embodiments, the optical measuring system 9 is arranged or positioned on the grain elevator 26. Generally speaking, the transparent window 16 may be adjacent to and/or border the harvested material transport path 5. In one or some embodiments, the transparent window 16 may be transparent for all wavelength ranges recorded by the optical measuring system 9. In one or some embodiments, the entire first and second field of vision 11, 14 may pass through the transparent window 16.

In one or some embodiments, the optical measuring system 9 has a light source 27. The light source 27 may be configured to emit light from some or all of the wavelength ranges recorded by the optical measuring system 9. In addition or alternatively, the light source 27 may be configured to sequentially emit light from some or all of the wavelength ranges recorded by the optical measuring system 9.

In one or some embodiments, the light source 27 irradiates the overlapping section U, and/or the first section A₁, and/or the second section A₂. In one or some embodiments, the light source 27 irradiates the particular section A₁, A₂ from the direction of the first and/or second optical sensor 10, 13. It therefore may be preferable for the particular sensor 10, 13 to record reflected light instead of transmitted light. In particular, it may also be provided for the light source 27 to be arranged or positioned outside of the first and/or second field of vision 11, 14.

As previously mentioned, the agricultural harvesting machine may include a grain elevator 26. In this case, any one, any combination, or all of the first optical sensor 10, the second optical sensor 13, the transparent window 16, the light source 27, or the housing 15 may be arranged or positioned on the grain elevator 26, such as under or over the grain elevator 26. Alternatively, any one, any combination, or all of the first optical sensor 10, the second optical sensor 13, the transparent window 16, the light source 27, or the housing 15 may be arranged or positioned behind the grain elevator 26, such as in the region of a grain tank filling system. Other possible arrangements, such as in a forage harvester in the region of a discharge chute, are also contemplated. An arrangement in front of the grain elevator 26 is also contemplated. In the last-cited case, the particular element(s) may be arranged behind the final threshing or the final separating work assembly 2.

In one or some embodiments, the overlapping section U may be part of a bottom side or top side of the harvested material flow, such as the main harvested material flow along the harvested material transport path 5.

In one or some embodiments, a method for operating a proposed agricultural harvesting machine is disclosed in which, using the agricultural production machine, a crop 3 is harvested and harvested material 4 is processed, wherein the measuring routine is performed using the optical measuring system 9, and wherein the analysis routine is performed using the evaluation device 12. Reference is made to all statements regarding the disclosed agricultural harvesting machine (including actions or steps performed by various parts of the disclosed agricultural harvesting machine).

In the method, the measuring routine and the analysis routine may be executed one or several times, such as iteratively or continuously. In one or some embodiments, the harvested material parameter may be determined in real time. The term “real time” may mean that only a predefined time span separates the recording or sensing of the particular image data and the determination of the harvested material parameter that, in one or some embodiments, may be less than one minute, may be less than 30 seconds, or may be less than five seconds. In response to the evaluation device determining the harvested material parameter(s), the evaluation device may cause the harvested material parameter(s) to be displayed to a user.

In particular within the context of the disclosed method, the monitoring assembly 8 may cyclically record a series of images. In one or some embodiments, within a predetermined processing time after the recording or sensing of a series of images, the evaluation device may then determine and display a harvested material parameter based on the series of images.

Further, it is intended that the foregoing detailed description be understood as an illustration of selected forms that the invention can take and not as a definition of the invention. It is only the following claims, including all equivalents, that are intended to define the scope of the claimed invention. Further, it should be noted that any aspect of any of the preferred embodiments described herein may be used alone or in combination with one another. Finally, persons skilled in the art will readily recognize that in preferred implementation, some, or all of the steps in the disclosed method are performed using a computer so that the methodology is computer implemented. In such cases, the resulting physical properties model may be downloaded or saved to computer storage.

### LIST OF REFERENCE NUMBERS

1 Combine
2 Work assembly
3 Crop
4 Harvested material
5 Harvested material transport path
6 Cutting unit
7 Grain tank
8 Monitoring assembly
9 Optical measuring system
10 First optical sensor
11 First field of vision
12 Evaluation device
13 Second optical sensor
14 Second field of vision
15 Housing
16 Transparent window
17 Inclined conveyor
18 Threshing concave
19 Threshing units
20 Diverter roller
21 Separating device
22 Returns pan
23 Cleaning device
24 Screening levels
25 Blower
26 Grain elevator
27 Light source
28 Processor
29 Memory
A₁ First section
A₂ Second section
U Overlapping section

1. An agricultural harvesting machine comprising:
at least one work assembly configured to harvest a crop and to transport harvested material from the crop in a harvested material flow along a harvested material transport path through the agricultural harvesting machine; and
a monitoring assembly including an optical measuring system positioned on at least a part of the harvested material transport path and an evaluation device configured to determine at least one harvested material parameter;
wherein the optical measuring system includes:
a first optical sensor positioned in the agricultural harvesting machine in order to generate spatially-resolved image data indicative of visible light from a visible wavelength range in a first field of vision thereby depicting the harvested material within a first section of the harvested material flow; and
a second optical sensor positioned in the agricultural harvesting machine in order to generate image data indicative of invisible light from an invisible wavelength range in a second field of vision thereby depicting the harvested material within a second section of the harvested material flow, wherein the first section and the second section at least partly overlap in an overlapping section; and
wherein the evaluation device is configured to:
correlate at least a part of the image data for the overlapping section from the first optical sensor and at least a part of the image data for the overlapping section from the second optical sensor; and
determine, based on the correlation, the at least one harvested material parameter.

2. The agricultural harvesting machine of claim 1, wherein the at least one harvested material parameter comprises at least one of composition of the harvested material or contents of the harvested material.

3. The agricultural harvesting machine of claim 1, wherein the at least one harvested material parameter comprises both of composition of the harvested material or contents of the harvested material.

4. The agricultural harvesting machine of claim 1, wherein the first optical sensor comprises an RGB camera; and
wherein the second optical sensor comprises a near-infrared spectrometer.

5. The agricultural harvesting machine of claim 1, wherein the overlapping section is formed by at least one of temporally or spatially.

6. The agricultural harvesting machine of claim 1, wherein the optical measuring system includes a housing and at least one transparent window; and
wherein the first optical sensor and the second optical sensor are positioned at least partly in the housing such that light from the harvested material passes through the at least one transparent window and is recorded by the first optical sensor and the second optical sensor.

7. The agricultural harvesting machine of claim 6, wherein the at least one transparent window is adjacent to or borders the harvested material transport path;
wherein the agricultural harvesting machine includes a grain elevator; and
wherein at least one of the first optical sensor, the second optical sensor, the at least one transparent window, a light source to irradiate the overlapping section, or the housing is positioned on the grain elevator.

8. The agricultural harvesting machine of claim 6, wherein both of the first optical sensor and the second optical sensor are positioned completely within the housing.

9. The agricultural harvesting machine of claim 1, wherein the evaluation device is configured to determine the at least one harvested material parameter relating to the overlapping section from the image data generated by the second optical sensor by the evaluation device determining a correction value from the image data of the first optical sensor and correcting the harvested material parameter using the correction value.

10. The agricultural harvesting machine of claim 9, wherein the correction value comprises a correction factor; and
wherein the evaluation device is configured to correct the harvested material parameter using the correction factor.

11. The agricultural harvesting machine of claim 9, wherein the at least one harvested material parameter relates to at least one of a grain portion, a broken grain portion, a non-grain portion, unthreshed components, straw, stem parts, silage, a content of the harvested material, starch content, or a grain moisture; and
wherein the correction value depicts a measurability of one or both of the first section or second section by the second optical sensor.

12. The agricultural harvesting machine of claim 1, wherein the first optical sensor is configured to generate spatially-resolved image data indicative of the visible light from at least two wavelength ranges within the first field of vision.

13. The agricultural harvesting machine of claim 1, wherein the first optical sensor is configured to generate spatially-resolved image data indicative of the visible light for an entire visible wavelength range within the first field of vision.

14. The agricultural harvesting machine of claim 1, wherein the first optical sensor comprises a line scan camera or area scan camera with sensor elements; and
wherein the sensor elements each record locally different pixels of the first field of vision that are at a distance from each other.

15. The agricultural harvesting machine of claim 1, wherein the second optical sensor has no more than 1000 sensor elements that record locally different pixels; and
wherein the second optical sensor is designed as a non-spatially resolved spectrometer.

16. The agricultural harvesting machine of claim 1, wherein at least one sensor element of the second optical sensor is configured to record invisible light from at least one wavelength range; and
wherein the at least one wavelength range is from an infrared range or an ultraviolet range.

17. The agricultural harvesting machine of claim 1, wherein the first section covers at least at least 90% of the second section.

18. The agricultural harvesting machine of claim 1, wherein the first section and the second section are coextensive.

19. A method for operating an agricultural harvesting machine comprising:
harvesting a crop and transporting harvested material from the crop in a harvested material flow along a harvested material transport path through the agricultural harvesting machine;
generating, using a first optical sensor, spatially-resolved image data indicative of visible light from a visible wavelength range in a first field of vision thereby depicting the harvested material within a first section of the harvested material flow;
generating, using a second optical sensor, image data indicative of invisible light from an invisible wavelength range in a second field of vision thereby depicting the harvested material within a second section of the harvested material flow, wherein the first section and the second section at least partly overlap in an overlapping section;
correlating at least a part of the image data for the overlapping section from the first optical sensor and at least a part of the image data for the overlapping section from the second optical sensor; and
determining, based on the correlation, at least one harvested material parameter.

20. The method of claim 19, wherein the overlapping section is formed by at least one of temporally or spatially.
